(19) United States
(12) Patent Application Publication
(10) Pub. No.: US 2007/0224684 A1
(43) Pub. Date: Sep. 27, 2007

(54) Transportable flow cytometer

(76) Inventors: David C. Olson, Ann Arbor, MI (US); Nathaniel C. Bair, Ann Arbor, MI (US); Richard L. Fisher, Ann Arbor, MI (US); Steve M. Martin, Ann Arbor, MI (US); Collin A. Rich, Ypsilanti, MI (US)

Correspondence Address: SCHOX PLC, 209 N. MAIN STREET #200, ANN ARBOR, MI, 48104, US

(21) Appl. No.: 11/387,186

(22) Filed: Mar. 22, 2006

(51) Int. Cl. G01N 33/48

(52) U.S. Cl. 436/63; 422/73

(57) ABSTRACT

A first preferred embodiment includes a flow cytometer with a fluidic system to draw a sample fluid into an interrogation zone, a light source to emit light toward the sample fluid in the interrogation zone, an optic system to collect and detect at least one of a scattered light and a fluorescent light from the interrogation zone, and a processor. The flow cytometer, if properly boxed and labeled, complies with the parcel post requirements of the United States Postal Service. A second preferred embodiment includes the method of supplying a flow cytometer by shipping the flow cytometer via the United States Postal Service. A third preferred embodiment includes the method of servicing a flow cytometer by receiving the flow cytometer from a user via the United States Postal Service and servicing the flow cytometer.

### TECHNICAL FIELD

This invention relates generally to the flow cytometer field, and more specifically to a transportable flow cytometer.

### BACKGROUND

In the flow cytometer market, there are broadly two types of flow cytometers: a handheld type that can be held and pocketed by a user, and a bench-top or floor mounted type that cannot be easily lifted and transported by a user. The handheld type, which is designed by Honeywell and Micronics and is often called a “lab card”, has been marketed as providing rapid, cost-effective results in infectious diseases testing, nucleic acid testing, blood type analysis, cancer testing, and respiratory disease testing. The typical lab cards, however, do not include a fluidic system to draw a sample fluid into an interrogation zone and, for this reason, are not considered appropriate for serious experiments in the lab.

The bench-top or floor-mounted type, which is sold by Becton Dickinson, typically include a fluidic system that draws sample fluid into the interrogation zone, which increases the reliability and speed of the flow cytometer and enables serious experiments. The typical bench-top or floor-mounted type, however, is a very large and very heavy machine and does not comply with the parcel post requirements of the United States Postal Service. Thus, when these machines fail and require repair, the machine cannot travel to a repair center, but rather the repair center must travel to the machine. This distributed service model requires training of skilled technicians and dispatching of mobile repair centers, which is potentially more expensive, less efficient, and less effective than the centralized service model.

Thus, there is a need in the flow cytometer field to create a transportable flow cytometer that includes a fluidic system that draws a sample fluid into an interrogation zone and complies with the parcel post requirements of the United States Postal Service. This invention provides such transportable flow cytometer.

### BRIEF DESCRIPTION OF THE FIGURES

FIG. 1 is a schematic representation of a first preferred embodiment of the invention.

FIGS. 2 and 3 are flowcharts of the second and third preferred embodiments of the invention, respectively.

FIG. 4 is a schematic representation of the fluidic system and the optic system of the first preferred embodiment.

FIGS. 5 and 6 are schematic representations of the optic systems of the first and second variations, respectively, of the first preferred embodiment.

FIG. 7 is a perspective view of the chassis of the first preferred embodiment.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

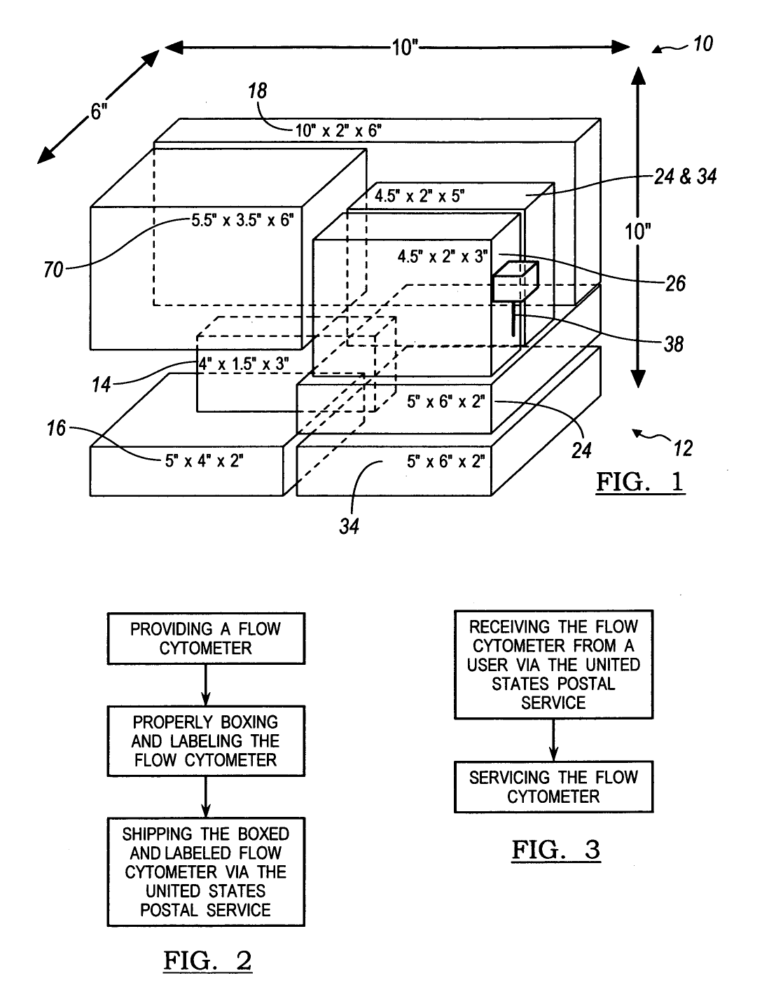

As shown in FIG. 1, a first preferred embodiment includes a flow cytometer 10 with a fluidic system 12 to draw a sample fluid into an interrogation zone, a light source 14 to emit light toward the sample fluid in the interrogation zone, an optic system 16 to collect and detect scattered and/or fluorescent light from the interrogation zone, and a processor 18. The interrogation zone functions to provide a location for the fluidic system 12 and the optic system 16 of the flow cytometer 10 to cooperatively facilitate the analysis of the sample fluid. The interrogation zone is preferably enclosed within a removable flow cell, but may alternatively be defined by any suitable system or device. The flow cytometer 10, if properly boxed and labeled, complies with the parcel post shipping requirements of the United States Postal Service. Through the novel selection of the components, the flow cytometer 10 transforms from a machine that is very large and very heavy which requires onsite repair, to a machine that can be easily lifted and transported which facilitates offsite repair. With the flow cytometer 10 of the first preferred embodiment, the overall total costs of ownership may be reduced, while offering greater freedom in mobility, placements, thermal management, venting options, and power consumption.

A second preferred embodiment, as shown in FIG. 2, includes the method of supplying a flow cytometer 10 by shipping the flow cytometer 10 via the United States Postal Service. The method preferably includes the following steps: (1) providing the flow cytometer 10 of the first preferred embodiment, (2) properly boxing and labeling the flow cytometer 10, and (3) shipping the boxed and labeled flow cytometer 10 via the United States Postal Service. The first step of the method may, however, include providing any suitable flow cytometer that draws a sample fluid into an interrogation zone. The second step preferably includes boxing the flow cytometer 10 in a conventional cardboard box, but may include boxing the flow cytometer 10 in any suitable container or may include labeling the chassis itself and shipping the chassis without any box. The third step of the method may include shipping the flow cytometer via any suitable standard carrier (such as DHL, FedEx, and UPS).

A third preferred embodiment, as shown in FIG. 3, includes a method of servicing a flow cytometer 10. The method preferably includes the following steps: (1) receiving the flow cytometer 10 from a user via the United States Postal Service; and (2) servicing the flow cytometer 10. The first step preferably includes receiving the flow cytometer 10 of the first preferred embodiment, but may alternatively include receiving any suitable flow cytometer that draws a sample fluid into an interrogation zone. Further, the first step may include receiving the flow cytometer 10 via any suitable standard carrier (such as DHL, FedEx, and UPS). The second step preferably includes conventional repair methods, but may alternatively include any suitable repair methods.

1. The Fluidic System

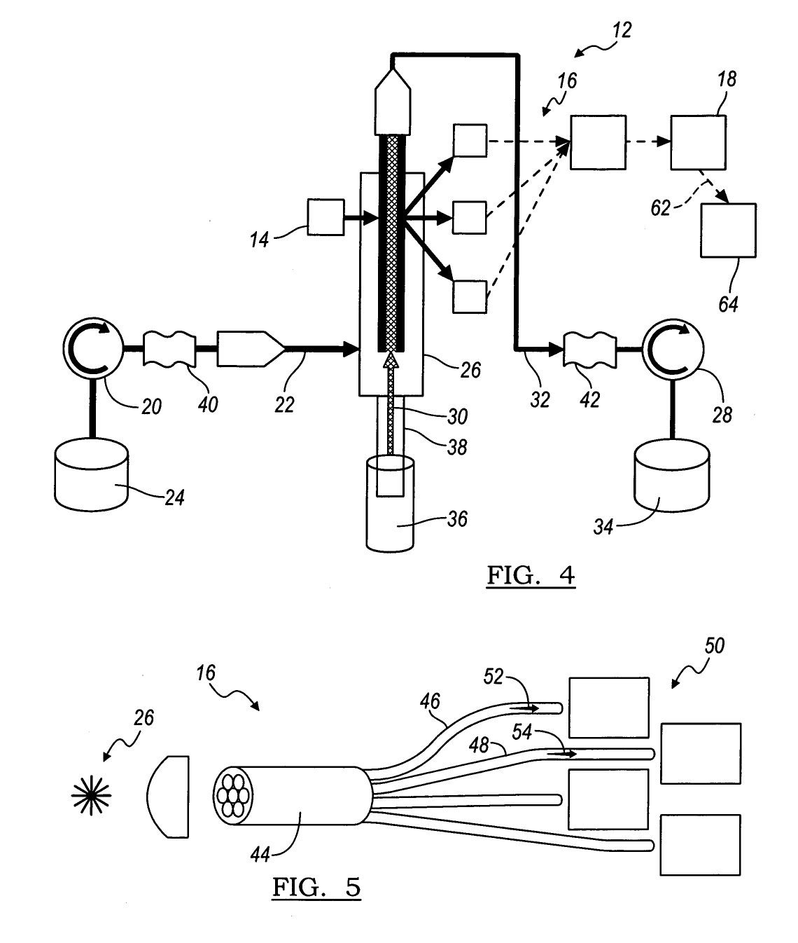

As shown in FIG. 4, the fluidic system 12 of the first preferred embodiment includes a sheath pump 20 to pump sheath fluid 22 from a sheath container 24 into an interrogation zone 26 and a waste pump 28 to pump the sheath fluid 22 and a sample fluid 30 as waste fluid 32 from the interrogation zone 26 into a waste container 34. The sheath pump 20 and/or the waste pump 28 draw sample fluid 30 from a sample container 36 into the interrogation zone 26. The fluidic system 12 is preferably the fluidic system described in U.S. patent application Ser. No. 11/370,714 entitled “Fluidic system for a Flow cytometer” and filed 8 Mar. 2006, which is hereby incorporated in its entirety by this reference. By using this fluidic system, the weight and size of the flow cytometer 10 may be reduced compared to other bench-top and floor-mounted type flow cytometers. The fluidic system 12 may, however, be any suitable fluidic system to draw a sample fluid into an interrogation zone.

The sheath pump 20 of the fluidic system 12 of the first preferred embodiment functions to pump sheath fluid 22 from the sheath container 24 into the interrogation zone 26. The sheath fluid 22 functions to hydrodynamically focus the sample fluid 30. The process of hydrodynamic focusing results in laminar flow of the sample fluid 30 within the flow cell and enables the optic system 16 to illuminate, and thus analyze, the particles within the sample fluid 30 with uniformity and repeatability. Preferably, the sheath fluid 22 is buffered saline or de-ionized water, but the sheath fluid 22 may alternatively be any suitable fluid to hydrodynamically focus the sample fluid 30. The sheath container 24 functions to contain the sheath fluid 22. The sheath container 24 is preferably a vented tank with a volume of approximately 1 Liter, but the sheath container 24 may alternatively be any suitable container to contain the sheath fluid 22. Preferably, the sheath pump 20 is a positive displacement pump. More preferably, the sheath pump 20 is a peristaltic pump with a flexible tube and one or more cams that pump the sheath fluid 22 through the flexible tube.

The waste pump 28 of the fluidic system 12 of the first preferred embodiment functions to pump the waste fluid 32 from the interrogation zone 26 into the waste container 34. Preferably, the waste fluid 32 includes the sheath fluid 22 and the sample fluid 30. Alternatively, the waste fluid 32 may include any fluid that exits the interrogation zone 26. The waste container 34 is preferably a vented tank with a volume of approximately 1 Liter, but the waste container 34 may alternatively be any suitable container to contain the waste fluid 32. Like the sheath pump 20, the waste pump 28 is preferably a positive displacement pump and more preferably a peristaltic pump with a flexible tube and one or more cams that pump the waste fluid 32 through the flexible tube.

The sheath pump 20 and the waste pump 28 of the fluidic system 12 of the first preferred embodiment cooperate to draw the sample fluid 30 from the sample container 36 and through a drawtube 38. The sample fluid 30 contains particles to be analyzed by the flow cytometer 10. The sample fluid 30 is preferably blood, but the sample fluid 30 may alternatively be any suitable fluid to be analyzed by the flow cytometer 10. The sample container 36, which functions to contain the sample fluid 30, is preferably an open beaker with a volume of approximately 5 milliliters, but may alternatively be any suitable container to contain the sample fluid 30. The drawtube 38, functions to convey the sample fluid 30 from the sample container 36 into the interrogation zone 26, is a conventional drawtube, but may alternatively be any suitable device to convey the sample fluid.

The sheath pump 20 and the waste pump 28 preferably cooperate to draw the sample fluid 30 from the sample container 36 into the interrogation zone 26 through the use of a pressure differential (e.g., the sheath pump 20 “pushes” the sheath fluid 22 and the waste pump 28 “pulls” the sheath fluid 22 and the sample fluid 30). In order to allow a variable flow rate of the sample fluid 30, the fluidic system 12 preferably allows for a variable flow rate of the sheath fluid 22 and/or the waste fluid 32. In a first variation, the sheath pump 20 and the waste pump 28 are driven by a single motor, but with a variable drive ratio device (e.g., transmission), such that the sheath pump 20 and the waste pump 28 may be operated at different pump speeds and, therefore, allow for a variable flow rate of the sheath fluid 22 and/or the waste fluid 32. In a second variation, the sheath pump 20 and the waste pump 28 are driven by a single motor, but the fluidic system 12 includes at least one by-pass valve located near the sheath pump 20 and/or the waste pump 28. The by-pass valve diverts a variable amount of the fluid flow and, therefore, allows for a variable flow rate of the sheath fluid 22 and/or waste fluid 32. In a third variation, the sheath pump 20 and the waste pump 28 are driven by a single motor, but the fluidic system 12 includes at least one restrictive valve located near the sheath pump 20 and/or the waste pump 28. The restrictive valve alters the fluid flow and, therefore, allows for a variable flow rate of the sheath fluid 22 and/or waste fluid 32. In a fourth variation, the sheath pump 20 and the waste pump 28 are driven by separate motors with separate controls and, therefore, allows for a variable flow rate of the sheath fluid 22 and/or waste fluid 32. The fluidic system 12 may, however, include other suitable variations that draw the sample fluid 30 from the sample container 36 into the interrogation zone 26 through the use of a pressure differential.

The fluidic system 12 of the first preferred embodiment also includes a first fluidic capacitor 40 located between the sheath container 24 and the interrogation zone 26 and a second fluidic capacitor 42 located between the interrogation zone 26 and the waste container 34. The fluidic capacitors 40 and 42 function to attenuate pulsations within the fluidic system 12. More specifically, the first fluidic capacitor 40 functions to temporarily expand/contract and thereby accumulate/release the sheath fluid 22 and attenuate pulsations within the sheath fluid 22. Similarly, the second fluidic capacitor 42 functions to temporarily expand/contract and thereby accumulate/release the waste fluid 32 and attenuate pulsations within the waste fluid 32. The fluidic capacitors 40 and 42 are selected from the group consisting of bellows-type with a diaphragm, bellows-type without a diaphragm, captive ball-type, and flexible tube-type. The fluidic capacitors 40 and 42 are preferably similar to the fluidic attenuators described in U.S. patent application Ser. No. 11/297,667 entitled “Pulsation Attenuator For A Fluidic system” and filed 7 Dec. 2005, which is hereby incorporated in its entirety by this reference. The fluidic capacitors 40 and 42 may, however, be any suitable device to attenuate pulsations within the fluidic system 12.

2. Light Source

The light source 14 of the first preferred embodiment functions to emit light toward the sample fluid 30 in the interrogation zone 26. The light source 14 is preferably a solid-state laser device. The wavelength emitted by the light source 14 is preferably 488 nm, but may be any suitable wavelength(s). By using this light source 14, the weight and size of the flow cytometer 10 may be reduced compared to other bench-top and floor-mounted type flow cytometers. The light source 14 may, however, be any suitable device or method to emit light toward the sample fluid 30 in the interrogation zone 26.

3. Optic System

The optic system 16 of the first preferred embodiment functions to collect and detect at least one of a scattered light and a fluorescent light from the interrogation zone 26. There are at least two preferred versions of the optic system 16. As shown in FIG. 5, the optic system 16 of a first version of the first preferred embodiment includes an optic device 44, a first waveguide 46, a second waveguide 48, and a detector subsystem 50. This first optic system 16 is preferably the optic system 16 described in U.S. patent application Ser. No. 11/297,170 entitled “System and Method for Guiding Light from an Interrogation zone to a Detector System” and filed 7 Dec. 2005, which is hereby incorporated in its entirety by this reference. By using this optic system, the weight and size of the flow cytometer 10 may be reduced compared to other bench-top and floor-mounted type flow cytometers. The first optic system 16 may, however, be any suitable fluidic system to collect and detect at least one of a scattered light and a fluorescent light from the interrogation zone.

The optic device 44 of the first optic system 16 functions to collect and partition light into a first channel 52 and a second channel 54 of substantially similar light from a substantially singular orientation of the interrogation zone 26. The first waveguide 46 functions to guide the first channel 52 from the optic device 44 to a detector system without substantial interruption. Likewise, the second waveguide 48 is functions to guide the second channel 54 from the optic device 44 to a detector system without substantial interruption. Preferably, the light of the first channel 52 can be filtered without affecting the light of the second channel 54, and the light of the second channel 54 can be filtered without affecting the light of the first channel 52. The detector subsystem 50 functions to measure the first channel 52 and the second channel 54. The detector subsystem 50 preferably includes a series of photodiodes, but may alternatively include a series of photomultiplier tubes (“PMT”) or any other suitable device:

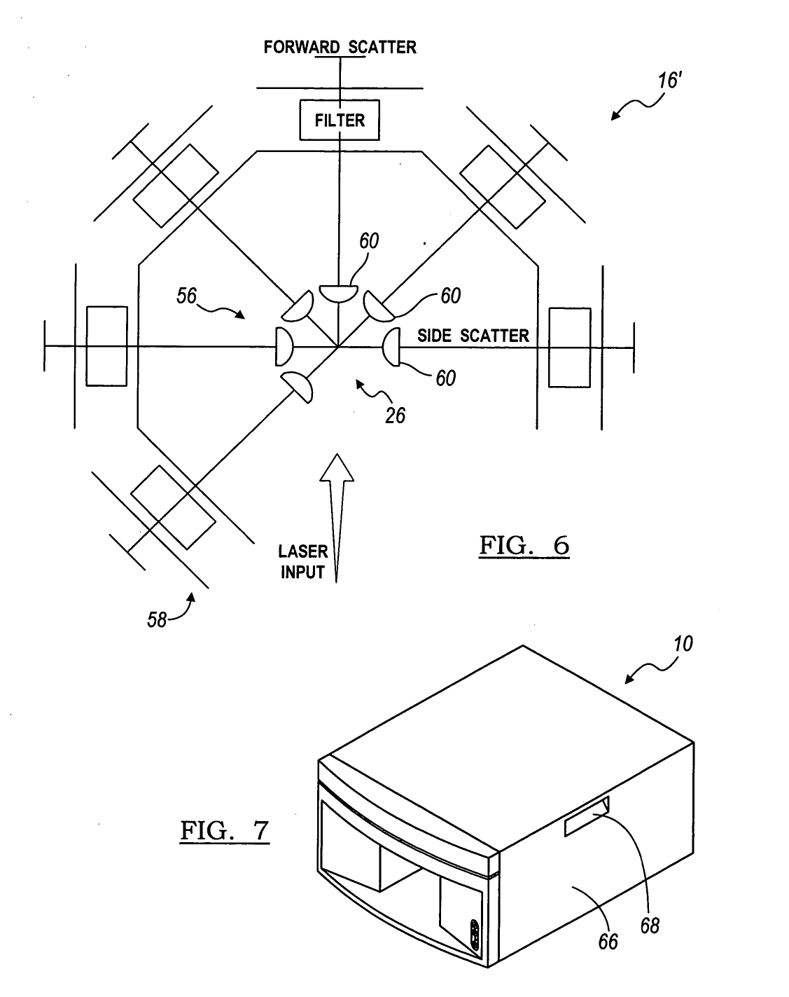

As shown in FIG. 6, the optic system 16′ of a second version of the first preferred embodiment includes a lens subsystem 56 with multiple lens surfaces arranged around the interrogation zone 26, and a detection subsystem 58 with multiple detectors arranged to detect the light collected and focused by the lens subsystem. This second optic system 16′ is preferably the optic system described in U.S. Patent Application Ser. No. 60/776,125 entitled “Multiple Path System for an Interrogation zone of a Flow cytometer” and filed 22 Feb. 2006, which is hereby incorporated in its entirety by this reference. By using this optic system, the weight and size of the flow cytometer 10 may be reduced compared to other bench-top and floor-mounted type flow cytometers. The second optic system 16 may, however, be any suitable optic system to collect and detect at least one of a scattered light and a fluorescent light from the interrogation zone.

The lens subsystem 56 of the second optic system 16′ functions to collect and focus the scattered and/or emitted light from the interrogation zone 26. The lens subsystem preferably includes at least three aspherical lenses 60. In one variation, the lenses 60 are truncated, which function to increase the light collecting ability of the lens subsystem 56, while maintaining a close proximity to the interrogation zone 26 and an overall compactness of the optic system 16. In other variations, the lenses 60 are not truncated, but rather placed very close together or formed as one piece. Preferably, the lens subsystem 56 includes at least three lens surfaces. More preferably, the lens subsystem 56 includes five or more lens surfaces. The lens subsystem is preferably arranged along a plane parallel to the light source 14 and perpendicular to the flow channel, but may alternatively be arranged in any suitable manner.

The detector subsystem 58 of the second optic system 16′ functions to detect light from the lens subsystem 56. The detector subsystem 58 preferably includes photosensor, such as a photomultiplier tube (“PMT”) or a photodiode, but may alternatively include any suitable device, such as a camera, to detect light or other electromagnetic energy. The detector subsystem 58 preferably includes a photosensor for every lens surface of the lens subsystem 56. The photosensors are preferably arranged in a direct path from the lens surfaces, and the light collected and directed by the lens subsystem 56 is preferably guided to the photosensors by an appropriate light path, such as an air channel.

4. Processor

As shown in FIG. 4, the processor 18 of the first preferred embodiment functions to control the light source 14 and to accept and process information from the optic system 16. The processor 18 preferably includes a first circuit board connected to the light source 14 and a second circuit board connected to the optic system 16. With this arrangement, the weight and size of the flow cytometer 10 of the first preferred embodiment may be reduced compared to other bench-top and floor-mounted type flow cytometers. The processor 18 may alternatively include any suitable device or method to control the light source 14 and to accept and process information from the optic system 16.

The flow cytometer 10 of the first preferred embodiment also includes an interface 62 connected to the processor 18. The interface 62 functions to communicate information between a host computer 64 (such as an iMac by the Apple Computer Company) and the processor 18. The interface 62 replaces the screen and keyboard of conventional flow cytometers and allows the weight and size of the flow cytometer 10 of the first preferred embodiment to be reduced compared to other bench-top and floor-mounted type flow cytometers. The interface 62 is preferably a wired USB interface, but may alternatively include a wireless USB interface or any other wired or wireless communication device or method that communicates information.

5. Other Elements

As shown in FIG. 7, the flow cytometer 10 of the first preferred embodiment also includes a chassis 66. The chassis 66 functions to contain the fluidic system, the light source, the optic system, and the processor, such that the entire flow cytometer 10 may be easily transported. The chassis 66 also functions to protect these elements during transportation. The chassis 66 is preferably made from a conventional plastic with conventional processes, but may be made from any suitable material and any suitable process. The chassis 66 of the first preferred embodiment includes at least one handle 68. The handle 68 functions to allow easily lifting of the flow cytometer 10. The chassis 66 preferably includes two handles 68, located on opposite sides of the flow cytometer 10, but the chassis 66 may include any suitable number of handles. The handle 68 is preferably integrated into the design of the chassis 66, but may be separately formed and attached to the chassis 66.

As shown in FIG. 1, the flow cytometer 10 of the first preferred embodiment also includes a power supply unit 70. The power supply unit 70 functions to transform power from a power grid in order to power the electrical components of the flow cytometer 10 (including the fluidic system 12, the light source 14, the optic system 16, and/or the processor 18). The power supply unit 70 is preferably entirely contained within the chassis 66 (shown in FIG. 7), but may be split between a first portion that is entirely contained within the chassis 66 and a second portion that, like a so-called “power brick” of an electronic device, is separate from the chassis 66. The power supply unit 70 preferably conforms to the AT power supply standard or the ATX power supply standard. More specifically, the power supply unit 70 measures 140 mm (approximately 5.5 inches) tall, by 150 mm (approximately 5.9 inches) wide, by 86 mm (approximately 3.4 inches) deep. By using a power supply unit 70 that conforms to these measurement standards, the size of the flow cytometer 10 of the first preferred embodiment may be reduced compared to other bench-top and floor-mounted type flow cytometers. The power supply unit 70 may alternatively be any suitable power supply unit that transform power from a power grid in order to power one or more electrical components of the flow cytometer.

6. Parcel Post Shipping Requirements

Through the novel combination of the fluidic system 12, the light source 14, the optic system 16, the processor 18, the chassis 66, and the power supply, the flow cytometer 10 of the first preferred embodiment is able to realize a significant reduction in the weight and size compared to other bench-top and floor-mounted type flow cytometers. More specifically, the flow cytometer 10 of the first preferred embodiment may be easily designed and manufactured with a weight equal to or less than 70 pounds and can be designed and manufactured with a weight equal to or less than 35 pounds. Further, the flow cytometer 10 of the first preferred embodiment may be easily designed and manufactured with a combined length and girth equal to or less than 130 inches and can be designed and manufactured with a combined length and girth equal to or less than 108 inches in combined length and girth.

According to the “Quick Service Guide 401” published by the United States Postal Service (which is incorporated in its entirety by this reference), if the flow cytometer 10 is properly boxed and labeled, measures equal to or less than 130 inches in combined length and girth, and weighs equal to or less than 35 pounds, the boxed and labeled flow cytometer 10 will qualify as parcel post oversized rate. Further, if the flow cytometer 10 is properly boxed and labeled, measures equal to or less than 108 inches in combined length and girth, and weighs equal to or less than 35 pounds, the boxed and labeled flow cytometer 10 will qualify as regular parcel post rate. These rates, parcel post oversized and parcel post, are the weight and measurement goals of the flow cytometer of the first preferred embodiment of the invention and, when realized, allow the flow cytometer 10 to be shipped via the United States Postal Service.

As a person skilled in the art will recognize from the previous detailed description and from the figures and claims, modifications and changes can be made to the preferred embodiments of the invention without departing from the scope of this invention defined in the following claims.

1. A flow cytometer to be used with a flow cell defining an interrogation zone comprising:
a fluidic system adapted to draw a sample fluid into the interrogation zone;
a light source adapted to emit light toward the sample fluid in the interrogation zone;
an optic system adapted to collect and detect at least one of a scattered light and a fluorescent light from the interrogation zone;
a processor coupled to the optic system; and
wherein the flow cytometer, if properly boxed and labeled, complies with the parcel post requirements of the United States Postal Service.

2. The flow cytometer of claim 1, wherein the fluidic system includes a sheath pump adapted to pump sheath fluid from a sheath container into the interrogation zone, a waste pump adapted to pump waste fluid from the interrogation zone into a waste container, wherein at least one of the sheath pump and the waste pump draw sample fluid from a sample container into the interrogation zone.

3. The flow cytometer of claim 2, wherein the sheath pump is a peristaltic pump.

4. The flow cytometer of claim 3, wherein the fluidic system also includes a fluidic capacitor located between the sheath container and the interrogation zone and adapted to temporarily expand and accumulate the sheath fluid to attenuate pulsations within the sheath fluid.

5. The flow cytometer of claim 1, wherein the light source is a solid-state laser device.

6. The flow cytometer of claim 1, wherein the optic system includes a lens system with at least three lens surfaces arranged around the interrogation zone, each lens surface adapted to collect at least one of a scattered light and a fluorescent light from the interrogation zone.

7. The flow cytometer of claim 6, wherein the lens surfaces are truncated aspherical lenses.

8. The flow cytometer of claim 7, wherein the optic system is a series of photomultiplier tube devices, each photomultiplier tube device having an integrated preamplifier.

9. The flow cytometer of claim 1, wherein the optic system includes an optical device adapted to collect and partition light into a first channel and a second channel, wherein the first channel and the second channel are substantially similar light from a substantially singular orientation of the interrogation zone; a first waveguide adapted to guide the first channel from the optical device to the light detector system without substantial interruption; and a second waveguide adapted to guide the second channel from the optical device to the light detector system without substantial interruption.

10. The flow cytometer of claim 1, wherein the optic system includes a series of photodiodes.

11. The flow cytometer of claim 1, wherein the processor includes a first circuit board connected to the light source and a second circuit board connected to the light detector.

12. The flow cytometer of claim 1, wherein the flow cytometer further includes an interface connected to the processor and adapted to communicate information between a host computer and the processor.

13. The flow cytometer of claim 1, further comprising a chassis, wherein the chassis substantially contains the optic system, the light source system, the fluidic system, the light detector, and the processor.

14. The flow cytometer of claim 13, further comprising a power supply unit adapted to transform power from a power grid, wherein the power supply unit is substantially contained within the chassis, and wherein the power supply unit substantially conforms to one of the AT and ATX power supply standards.

15. The flow cytometer of claim 1, wherein the flow cytometer weighs equal to or less than 70 pounds.

16. The flow cytometer of claim 15, wherein the flow cytometer weighs equal to or less than 35 pounds.

17. The flow cytometer of claim 1, wherein the flow cytometer measures equal to or less than 130 inches in combined length and girth.

18. The flow cytometer of claim 17, wherein the flow cytometer measures equal to or less than 108 inches in combined length and girth.

19. A method of supplying a flow cytometer, comprising the following steps:
providing a flow cytometer to be used with a flow cell defining an interrogation zone, wherein the flow cytometer includes a fluidic system adapted to draw a sample fluid into the interrogation zone, a light source adapted to emit light toward the sample fluid in the interrogation zone, an optic system adapted to collect and detect at least one of a scattered light and a fluorescent light from the interrogation zone, and a processor coupled to the optic system;
properly boxing and labeling the flow cytometer; and
shipping the boxed and labeled flow cytometer via the United States Postal Service.

20. A method of servicing a flow cytometer having a fluidic system, a light source, an optic system, and a processor, the method comprising the following steps:
receiving the flow cytometer from a user via the United States Postal Service; and
servicing the flow cytometer.

21. The flow cytometer of claim 13, further comprising a handle adapted to facilitate lifting of the flow cytometer.

22. The flow cytometer of claim 13, further comprising a power supply unit adapted to transform power from a power grid and having a first portion that is contained within the chassis and a second portion that is separate from the chassis.
